(19) United States
(12) Patent Application Publication
(10) Pub. No.: US 2025/0352177 A1
(43) Pub. Date: Nov. 20, 2025

(54) BACKING MATERIAL, ULTRASOUND PROBE, ULTRASOUND DIAGNOSTIC APPARATUS, AND CURABLE RESIN COMPOSITION

(71) Applicant: FUJIFILM Corporation, Tokyo (JP)

(72) Inventors: Kazushi Furukawa, Ashigarakami-gun (JP); Yoshihiro Nakai, Ashigarakami-gun (JP)

(73) Assignee: FUJIFILM Corporation, Tokyo (JP)

(21) Appl. No.: 19/290,600

(22) Filed: Aug. 5, 2025

Related U.S. Application Data
(63) Continuation of application No. PCT/JP2024/012827, filed on Mar. 28, 2024.

(30) Foreign Application Priority Data
Mar. 29, 2023 (JP) 2023-052936

(51) Int. Cl. A61B 8/00; B29C 43/00; B29K 63/00

(52) U.S. Cl. CPC A61B 8/4455; B29C 43/003; B29K 2063/00

(57) ABSTRACT

Provided are a backing material, an ultrasound probe, an ultrasound diagnostic apparatus, and a curable resin composition. A backing material for an ultrasound probe includes thermally conductive particles and a resin, in which the resin includes any one of the following (A) to (C): (A) a reaction cured product of an epoxy resin having a polyurethane structure and a polyamine compound; (B) a reaction cured product of an epoxy resin and a polyamine compound, the reaction cured product having a polyether structure; and (C) a reaction cured product of a polyisocyanate compound and a polyamine compound, a loss tangent of the resin in a range of 0° C. to 50° C. is 0.06 or more, a loss tangent of the resin in a range of −20° C. to 110° C. is less than 1.50, a storage elastic modulus of the backing material in a range of 0° C. to 50° C. is 1000 MPa or more, and a content of the resin in the backing material is 25% to 50% by volume.

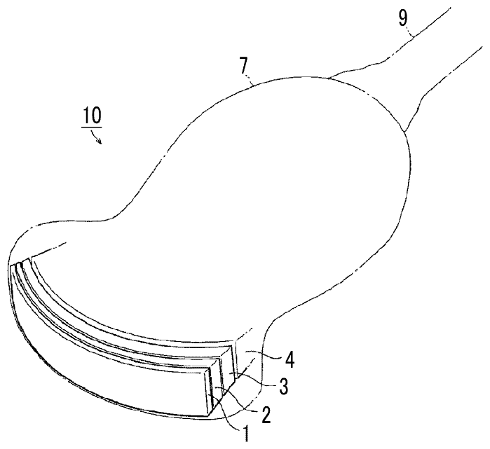

### CROSS-REFERENCE TO RELATED APPLICATIONS

This application is a Continuation of PCT International Application No. PCT/JP2024/012827 filed on Mar. 28, 2024, which claims priority under 35 U.S.C. § 119(a) to Japanese Patent Application No. 2023-052936 filed in Japan on Mar. 29, 2023. The above application is hereby expressly incorporated by reference, in its entirety, into the present application.

### BACKGROUND OF THE INVENTION

### 1. Field of the Invention

The present invention relates to a backing material, an ultrasound probe, an ultrasound diagnostic apparatus, and a curable resin composition.

### 2. Description of the Related Art

In an ultrasound measurement device, an ultrasonic probe is used that irradiates a test target or a part (hereinafter, simply referred to as an object) with ultrasonic waves, receives reflected waves (echoes), and outputs a signal. The reflected wave received by the ultrasound probe is converted into an electric signal, and the electric signal is displayed as an image. As a result, an inside of the test object is visualized and observed.

For example, in the ultrasound probe, during diagnosis, an acoustic lens of the ultrasound probe is brought into contact with a subject, and a piezoelectric element is driven to transmit an ultrasound signal into the subject from a front surface of the piezoelectric element. The ultrasound signal is focused on a required position in the subject by electronic focusing based on the driving timing of the piezoelectric element and focusing by the acoustic lens. In this case, the driving timing of the piezoelectric element can be controlled to transmit the ultrasound signal to a required range in the subject, and reception processing is performed on an echo signal from the subject to obtain an ultrasound image (tomographic image) of the required range. The ultrasound signal is also emitted to a rear surface by the driving of the piezoelectric element. Therefore, a backing member is disposed on the rear surface of the piezoelectric element, and the ultrasound signal emitted to the rear surface is absorbed (attenuated) by the backing material. Therefore, the adverse effect of a normal ultrasound signal being transmitted into the subject along with the ultrasound signal (reflected signal) from the rear surface side is avoided.

The backing member is required to have all of the following properties, in addition to the high ultrasound attenuation properties: high heat dissipation properties capable of efficiently dissipating heat emitted from the piezoelectric element during the driving of the ultrasound probe; and workability that enables obtaining the backing member for the ultrasound probe with high productivity while suppressing the occurrence of deformation, breakage, and the like.

A technique has been known that adds a thermally conductive filler to impart high heat dissipation properties to the backing member.

For example, JP2006-033801A discloses an acoustic backing composition containing an ethylene-vinyl acetate copolymer having a vinyl acetate content of 20% to 80% by weight and a thermally conductive filler. In addition, JP2000-165995A discloses a material that contains a main material consisting of nitrile rubber, butyl rubber, or urethane rubber and a thermally conductive filler consisting of aluminum nitride, silicon carbide, copper, boron nitride, or graphite as a rear surface load member (backing member) for an ultrasound probe.

### SUMMARY OF THE INVENTION

The results of studies conducted by the present inventors proved that, in the acoustic backing composition disclosed in JP2006-033801A, the acoustic backing member having both high heat dissipation properties and high ultrasound attenuation properties was obtained, but the strength was insufficient due to the softness of the ethylene-vinyl acetate copolymer, making processing difficult and posing problems for the use of the acoustic backing member as a backing member. In addition, the rear surface load member (backing member) disclosed in JP2000-165995A also has both high heat dissipation properties and high ultrasound attenuation properties. However, it is difficult to process the rear surface load member due to the softness and poor workability of the rubber material, and there are still problems with using the rear surface load member as the backing material.

An object of the present invention is to provide a backing material whose heat dissipation properties have been improved by mixing of thermally conductive particles and which has both excellent ultrasound attenuation properties and excellent workability, and an ultrasound probe and an ultrasound diagnostic apparatus including the backing material. In addition, an object of the present invention is to provide a curable resin composition suitable for forming the backing material.

The above-mentioned objects of the present invention have been achieved by the following means.
[1] There is provided a backing material for an ultrasound probe comprising: thermally conductive particles; and a resin, in which the resin includes any one of the following (A) to (C): (A) a reaction cured product of an epoxy resin having a polyurethane structure and a polyamine compound; (B) a reaction cured product of an epoxy resin and a polyamine compound, the reaction cured product having a polyether structure; and (C) a reaction cured product of a polyisocyanate compound and a polyamine compound, a loss tangent of the resin in a range of 0° C. to 50° C. is 0.06 or more, a loss tangent of the resin in a range of −20° C. to 110° C. is less than 1.50, a storage elastic modulus of the backing material in a range of 0° C. to 50° C. is 1000 MPa or more, and a content of the resin in the backing material is 25% to 50% by volume.
[2] In the backing material according to [1], the thermally conductive particles include at least one of metal particles or ceramic particles.
[3] There is provided an ultrasound probe comprising the backing material according to [1] or [2].
[4] There is provided an ultrasound diagnostic apparatus using the ultrasound probe according to [3].
[5] There is provided a curable resin composition for forming the backing material according to [1] or [2], the curable resin composition comprising: the thermally conductive particles; and any one of the following (a) to (c) as a resin component: (a) a combination of an epoxy resin having a polyurethane structure and a polyamine compound; (b) a combination of an epoxy resin and a polyamine compound, at least one of the epoxy resin or the polyamine compound having a polyether structure; and (c) a combination of a polyisocyanate compound and a polyamine compound, in which a viscosity of the curable resin composition is 5000 Pa·sec or less under conditions of 25° C. and a shear rate of 0.01/sec.

In the present invention, any numerical range represented by using “to” refers to a range including numerical values described before and after “to” as a lower limit value and an upper limit value, respectively.

The backing material according to the present invention is a backing material having heat dissipation properties achieved by the mixing of thermally conductive particles and has excellent ultrasound attenuation properties and workability.

In addition, the ultrasound probe and the ultrasound diagnostic apparatus according to the present invention include the backing material having the above-described performance.

Further, the curable resin composition according to the present invention is suitable for forming the above-described backing material.

### BRIEF DESCRIPTION OF THE DRAWINGS

The FIGURE is a perspective view showing an example of a convex-type ultrasound probe which is an aspect of an ultrasound probe.

### DESCRIPTION OF THE PREFERRED EMBODIMENTS

### Backing Material

A backing material according to the present invention is a backing material for an ultrasound probe including thermally conductive particles and a resin. The resin includes any one of the following (A) to (C): (A) a reaction cured product of an epoxy resin having a polyurethane structure and a polyamine compound; (B) a reaction cured product of an epoxy resin and a polyamine compound, the reaction cured product having a polyether structure; and (C) a reaction cured product of a polyisocyanate compound and a polyamine compound. A loss tangent of the resin in a range of 0° C. to 50° C. is 0.06 or more, and a loss tangent of the resin in a range of −20° C. to 110° C. is less than 1.50. A storage elastic modulus of the backing material in a range of 0° C. to 50° C. is 1000 MPa or more, and a content of the resin in the backing material is 25% to 50% by volume.

The details of the reason why the backing material according to the present invention has excellent ultrasound attenuation properties and workability are not clear, but are considered as follows.

Since the backing material according to the present invention is a backing material for an ultrasound probe including thermally conductive particles and a resin having specific viscoelastic properties, it is possible to exhibit excellent ultrasound attenuation properties and excellent workability while exhibiting high heat dissipation properties achieved by the thermally conductive particles. Specifically, since the resin has a loss tangent of 0.06 or more in the range of 0° C. to 50° C., it is possible to exhibit excellent ultrasound attenuation properties. It is considered that the reason is that, in a case where ultrasonic waves are emitted to the resin having these properties, polymer molecular chains constituting the resin absorb the energy of vibrations and consume the energy as their own kinetic energy, resulting in the disappearance of the ultrasonic waves. In addition, since the resin has a loss tangent of less than 1.50 in the range of −20° C. to 110° C., softening or the like is less likely to occur even though the temperature of the resin is increased during processing such as dicing. Further, since the backing material has a storage elastic modulus of 1000 MPa or more in the range of 0° C. to 50° C., positional deviation or the like is less likely to occur due to stress during processing, and it is possible to exhibit excellent workability.

The present inventors have also found that the resin having the above-described specific viscoelastic properties can be achieved by using a specific amount of resin selected from (A) the reaction cured product of an epoxy resin having a polyurethane structure and a polyamine compound, (B) the reaction cured product of an epoxy resin and a polyamine compound which has a polyether structure, and (C) the reaction cured product of a polyisocyanate compound and a polyamine compound. The reason why the resin including any one of (A) to (C) has the above-described specific viscoelastic properties is not clear, but it is considered that the following is related to this: the reaction cured products (A) to (C) have numerous polar functional groups (urethane bonds, ether bonds, and urea bonds) within the molecular chains, which results in strong intermolecular interactions.

Hereinafter, the backing material according to the present invention will be described in detail.

### (Loss Tangent of Resin)

The resin contained in the backing material according to the present invention has a loss tangent of 0.06 or more in a range of 0° C. to 50° C. and has a loss tangent of less than 1.50 in a range of −20° C. to 110° C.

The loss tangent being 0.06 or more in the range of 0° C. to 50° C. means that a minimum value of the loss tangent in the range of 0° C. to 50° C. is 0.06 or more.

The minimum value of the loss tangent in the range of 0° C. to 50° C. is not particularly limited as long as the minimum value is 0.06 or more and is usually 0.40 or less. The minimum value of the loss tangent in the range of 0° C. to 50° C. may be in a range of 0.06 to 0.40, preferably in a range of 0.06 to 0.30, more preferably in a range of 0.06 to 0.20, still more preferably in a range of 0.06 to 0.15, and particularly preferably in a range of 0.06 to 0.12.

The loss tangent being less than 1.50 in the range of −20° C. to 110° C. means that a maximum value of the loss tangent in the range of −20° C. to 110° C. is less than 1.50.

The maximum value of the loss tangent in the range of −20° C. to 110° C. is not particularly limited as long as the maximum value is less than 1.50 and is usually 0.10 or more. The maximum value of the loss tangent in the range of −20° C. to 110° C. may be in a range of 0.10 to 1.50, is preferably in a range of 0.10 to 1.25, more preferably in a range of 0.10 to 1.00, still more preferably in a range of 0.10 to 0.75, and particularly preferably in a range of 0.10 to 0.55.

In addition, the loss tangent of the resin is measured by a method described in Examples which will be described below. It is assumed that the “loss tangent in the range of 0° C. to 50° C.” and the “loss tangent in the range of −20° C. to 110° C.” are values obtained by rounding off the measured values to two decimal places.

### (Storage Elastic Modulus of Backing Material)

The storage elastic modulus of the backing material according to the present invention in the range of 0° C. to 50° C. is 1000 MPa or more.

The storage elastic modulus being 1000 MPa or more in the range of 0° C. to 50° C. means that a minimum value of the storage elastic modulus in the range of 0° C. to 50° C. is 1000 MPa or more.

The minimum value of the storage elastic modulus in the range of 0° C. to 50° C. is preferably 2000 MPa or more, more preferably 2500 MPa or more, still more preferably 3000 MPa or more, and particularly preferably 3300 MPa or more.

The minimum value of the storage elastic modulus in the range of 0° C. to 50° C. is usually less than 8000 MPa. That is, the range of the minimum value of the storage elastic modulus in the range of 0° C. to 50° C. may be 1000 MPa or more and less than 8000 MPa, is preferably 2000 MPa or more and less than 8000 MPa, more preferably 2500 MPa or more and less than 8000 MPa, still more preferably 3000 MPa or more and less than 8000 MPa, and particularly preferably 3300 MPa or more and less than 8000 MPa.

In addition, the storage elastic modulus of the backing material is measured by a method described in Examples which will be described below.

### (Attenuation Rate of Backing Material)

An attenuation rate of the backing material according to the present invention is preferably more than 0.8 dB/ (mm·MHz) , more preferably more than 2.5 dB/(mm·MHz), still more preferably more than 3.0 dB/(mm·MHz), particularly preferably more than 3.5 dB/(mm·MHz), and most preferably more than 4.0 dB/(mm·MHz).

In addition, the attenuation rate of the backing material is measured by a method described in Examples which will be described below.

### <Resin>

The resin contained in the backing material according to the present invention includes any one of the following (A) to (C):
(A) A reaction cured product of an epoxy resin having a polyurethane structure and a polyamine compound;
(B) A reaction cured product of an epoxy resin and a polyamine compound which has a polyether structure; and
(C) A reaction cured product of a polyisocyanate compound and a polyamine compound.

Further, in a case where the reaction cured product of an epoxy resin having a polyurethane structure and a polyamine compound has a polyether structure, the reaction cured product is classified as (A) instead of (B). Therefore, the epoxy resin in the reaction cured product (B) does not have a polyurethane structure.

In addition, even in a case where the reaction cured product of a polyisocyanate compound and a polyamine compound has a polyether structure, the reaction cured product is classified as (C).

From the viewpoint of further improving workability, it is preferable that the resin contained in the backing material according to the present invention includes (C).

### (A) Reaction Cured Product of Epoxy Resin Having Polyurethane Structure and Polyamine Compound

### (Epoxy Resin Having Polyurethane Structure)

The epoxy resin having a polyurethane structure is not particularly limited and can be used as long as the epoxy resin has a polyurethane structure and an epoxy group. The number of epoxy groups contained in the epoxy resin having a polyurethane structure is usually two or more, preferably two to four, and more preferably two or three. For example, an epoxy resin with a polyurethane structure that has a number-average molecular weight of 200 to 20000 is generally used as a commercially available epoxy resin having a polyurethane structure. Specifically, the following can be given as examples of the epoxy resin:

ADEKA RESIN EPU-6, ADEKA RESIN EPU-7N, ADEKA RESIN EPU-11F, ADEKA RESIN EPU-15F, ADEKA RESIN EPU-1395, ADEKA RESIN EPU-73B, ADEKA RESIN EPU-17, ADEKA RESIN EPU-17T-6, and ADEKA RESIN EPU-1001 (all of which are product names, manufactured by ADEKA Corporation), EPOXY 802-30CX, EPOXY 803, EPOXY 820-40CX, EPOXY 830, EPOXY 834, EPOXY 840, EPOXY 815, EPOXY 837, EPOXY 810ST, and EPOXY 505-15 (all of which are product names, manufactured by Mitsui Chemicals, Inc.), and the like.

Among these materials, ADEKA RESIN EPU-7N, ADEKA RESIN EPU-11F, or EPU-17 (all produce names, manufactured by ADEKA Corporation) is preferable since the material has excellent mixability with thermally conductive particles.

The epoxy group equivalent of the epoxy resin having a polyurethane structure is not particularly limited and is, for example, preferably 170 to 2000 g/mol and more preferably 200 to 500 g/mol. In addition, the epoxy group equivalent means the mass (g) of the epoxy resin having a polyurethane structure per 1 mol of epoxy groups.

A viscosity of the epoxy resin having a polyurethane structure at 25° C. is not particularly limited and is, for example, preferably 200 to 200000 mPa·sec and more preferably 600 to 30000mPa·sec. Further, the viscosity is a value measured by a method described in Examples which will be described below. In addition, the same applies to the description of the viscosity in the following (A) to (C).

### (Polyamine Compound)

The polyamine compound that is reacted with the epoxy resin having a polyurethane structure is not particularly limited and can be used as long as the polyamine compound has two or more amino groups. A polyamine compound generally used as a curing agent for an epoxy resin is preferably used.

The polyamine compound may be any of an aliphatic polyamine compound {a chain-like aliphatic polyamine compound in which an amino group is bonded to an aliphatic chain (however, the chain-like aliphatic polyamine compound does not have a ring structure), a cyclic aliphatic polyamine compound in which an amino group is bonded to an aliphatic ring directly or via an aliphatic chain (however, the cyclic aliphatic polyamine compound may have a nitrogen atom constituting an amino group as a ring-constituting atom of an aliphatic ring), and an aliphatic polyamine compound having an aromatic ring (an aliphatic polyamine compound in which an amino group is bonded to an aliphatic chain or an aliphatic ring and which has an aromatic ring)} or an aromatic polyamine compound (a polyamine compound in which an amino group is directly bonded to an aromatic ring), or a mixture thereof. The aliphatic polyamine compound is preferable since the aliphatic polyamine compound has excellent reactivity. The polyamine compound may have a ring structure as described above. Further, the polyamine compound may contain a heteroatom, such as an oxygen atom or a sulfur atom, in addition to the nitrogen atom and may have a polyether structure. A preferred example of the polyether structure is a polyether structure having a number-average molecular weight of 200 to 6000.

The number of amino groups in the polyamine compound is preferably two or three.

Two or more amino groups contained in the polyamine compound may be any amino groups having active hydrogen and may be, specifically, at least one of an unsubstituted amino group (—NH₂) or a monosubstituted amino group having one active hydrogen. The unsubstituted amino group (—NH₂) is preferable. Further, the monosubstituted amino group having one active hydrogen may be incorporated into the compound in the form of >NH. In addition, the polyamine compound may have a disubstituted amino group (an amino group that does not have active hydrogen) in addition to the two or more amino groups (amino groups having active hydrogen) contained in the polyamine compound.

The number of active hydrogens derived from the amino groups contained in the polyamine compound may be two or more and is preferably three to six and more preferably four to six.

Among these amino groups, the polyamine compound preferably has two or more unsubstituted amino groups (—NH₂) and more preferably has two or three unsubstituted amino groups (—NH₂).

The polyamine compound may be a low-molecular-weight compound or a high-molecular-weight compound.

Specific examples of the polyamine compound include the following.

Examples of the chain-like aliphatic polyamine compound include diethylenetriamine, triethylenetetramine, tetraethylenepentamine, dipropylenediamine, diethylaminopropylamine, hexamethylenediamine, and 2,2,4-trimethylhexamethylenediamine. In addition, examples of the chain-like aliphatic polyamine compound containing an oxygen atom include chain-like aliphatic polyamine compounds having a polyalkylene oxide structure, such as a polyethylene oxide structure or a polypropylene oxide structure. Examples of the chain-like aliphatic polyamine compound can include JEFFAMINE D-230, JEFFAMINE D-400, JEFFAMINE D-2000, JEFFAMINE T-403, and JEFFAMINE T-5000 (all of which are product names, manufactured by HUNTSMAN Corporation).

Further, examples of the cyclic aliphatic polyamine compound include N-aminoethylpiperazine, 4,4′-methylenebis (2-methylcyclohexane-1-amine), mensendiamine, isophoronediamine, bis(4-aminocyclohexyl)methane, and 1,3-bisaminomethylcyclohexane.

Furthermore, examples of the aliphatic polyamine compound having an aromatic ring (the polyamine compound in which an amino group is bonded to an aliphatic chain or an aliphatic ring and which has an aromatic ring) include m-xylylenediamine, Gaskamine 240, and Gaskamine 328 (all of which are product names, manufactured by Mitsubishi Gas Chemical Company, Inc.).

Moreover, examples of the aromatic polyamine compound include m-phenylenediamine, diaminodiphenylmethane, and diaminodiphenyl sulfone. In addition, examples of the aromatic polyamine compound can include Elasmer 250P, Elasmer 650P, Elasmer 1000P, and Polyurea SL-100A (all of which are product names, manufactured by Kumiai Chemical Co., Ltd.).

Among these polyamine compounds, it is preferable to include the aliphatic polyamine compound having an aromatic ring since the aliphatic polyamine compound has good reactivity and excellent intermolecular interaction. In particular, from the viewpoint of further improving ultrasound attenuation properties and workability in the form including the reaction cured product (A), it is preferable to contain the aliphatic polyamine compound having an aromatic ring and the chain-like aliphatic polyamine compound having no aromatic ring.

The amino group equivalent of the polyamine compound is not particularly limited and is, for example, preferably 30 to 12000 g/mol, more preferably 30 to 3000 g/mol, and still more preferably 30 to 1200 g/mol. In addition, the amino group equivalent means the mass (g) of the polyamine compound per 1 mol of amino groups.

The polyamine compound may be a solid or a liquid at 25° C. In a case where the polyamine compound is a liquid at 25° C., the viscosity of the polyamine compound at 25° C. is not particularly limited and is, for example, preferably 1 to 30000 mPa·sec, more preferably 3 to 30000 mPa·sec, and still more preferably 3 to 20000 mPa·sec.

### (B) Reaction Cured Product of Epoxy Resin and Polyamine Compound Which Has Polyether Structure

A reaction cured product of an epoxy resin and a polyamine compound having two or more amino groups can be used as the reaction cured product (B), without particular limitation, as long as the reaction cured product has a polyether structure.

The reaction cured product (B) can be obtained by any one of a reaction between an epoxy resin having a polyether structure and a polyamine compound having no polyether structure, a reaction between an epoxy resin having no polyether structure and a polyamine compound having a polyether structure, or a reaction between an epoxy resin having a polyether structure and a polyamine compound having a polyether structure. In general, the polyether structure of the reaction cured product obtained in this way is a polyether structure having a number-average molecular weight of 200 to 6000. Among these reaction cured products, the reaction cured product of (B) is preferably a reaction cured product of an epoxy resin having a polyether structure and a polyamine compound having no polyether structure or a reaction cured product of an epoxy resin having no polyether structure and a polyamine compound having a polyether structure. From the viewpoint of exhibiting a more preferable viscosity as a curable resin composition, it is more preferable that the reaction cured product of (B) is a reaction cured product of an epoxy resin having no polyether structure and a polyamine compound having a polyether structure.

### (Epoxy Resin)

The number of epoxy groups contained in the epoxy resin is usually two or more, preferably two to four, and more preferably two or three.

For example, an epoxy resin having a polyether structure with a number-average molecular weight of 200 to 6000 is generally used as a commercially available epoxy resin having a polyether structure. Specifically, the following can be given as examples of the commercially available epoxy resin having a polyether structure:

DENACOL EX-851, DENACOL EX-832, DENACOL EX-101, DENACOL EX-103 (all of which are product names, manufactured by Nagase ChemteX Corporation), DER 732 (product name, manufactured by Dow Chemical Company), ADEKA RESIN EP-4000 (product name, manufactured by ADEKA Corporation), and the like.

From the viewpoint of excellent mechanical strength, it is preferable to have a bisphenol structure, and ADEKA RESIN EP-4000, DENACOL EX-103, and the like are preferable.

Examples of the epoxy resin that does not have a polyether structure include a bisphenol A-type epoxy resin, a bisphenol F-type epoxy resin, a bisphenol E-type epoxy resin, and a novolac-type epoxy resin. From the viewpoint of excellent mechanical strength of the obtained reaction cured product, the bisphenol A-type epoxy resin is preferable.

The epoxy group equivalent of the epoxy resin is not particularly limited and is, for example, preferably 160 to 550 g/mol, more preferably 160 to 400 g/mol, and still more preferably 165 to 230 g/mol. In addition, the epoxy group equivalent means the mass (g) of the epoxy resin per 1 mol of epoxy groups.

The viscosity of the epoxy resin at 25° C. is not particularly limited and is, for example, preferably 700 to 20000 mPa·sec, more preferably 1000 to 20000 mPa·sec, and still more preferably 1000 to 15000 mPa·sec.

### (Polyamine Compound)

A polyamine compound that is reacted with the epoxy resin having a polyurethane structure in (A) can be given as an example of the polyamine compound for obtaining the reaction cured product (B), and the preferred range of the polyamine compound is the same as described above.

For example, a polyamine compound having a polyether structure with a number-average molecular weight of 200 to 6000 is generally used as the commercially available polyamine compound having a polyether structure. Specifically, examples of the polyamine compound are as follows.

Among the polyamine compounds that are reacted with the epoxy resin having a polyurethane structure in (A), for example, JEFFAMINE D-230, JEFFAMINE D-400, JEFFAMINE D-2000, JEFFAMINE T-403, JEFFAMINE T-5000 (all of which are product names, manufactured by HUNTSMAN Corporation) and the like can be preferably used. It is also preferable to use a mixture of a plurality of types of compounds in order to adjust the average molecular weight and to adjust the mechanical strength of the obtained reaction cured product. For example, a mixture of JEFFAMINE D-400 and JEFFAMINE D-2000 or a mixture of JEFFAMINE D-230 and JEFFAMINE T-5000, all of which are product names manufactured by HUNTSMAN Corporation, can be used to obtain suitable mechanical strength.

### (C) Reaction Cured Product of Polyisocyanate Compound and Polyamine Compound

### (Polyisocyanate Compound)

The polyisocyanate compound is not particularly limited, and any polyisocyanate compound can be used as long as the polyisocyanate compound has two or more isocyanato groups. For example, various polyisocyanate compounds used for manufacturing polyurethane and polyurea can be preferably used.

The polyisocyanate compound may be any of an aliphatic isocyanate compound (a compound in which an isocyanato group is bonded to an aliphatic chain or an aliphatic ring) or an aromatic isocyanate compound (a compound in which an isocyanato group is bonded to an aromatic ring) and may be a mixture thereof. The polyisocyanate compound may have a ring structure.

The number of isocyanato groups in the polyisocyanate compound is preferably two or three and more preferably two.

Examples of the polyisocyanate compound can include diphenylmethane diisocyanate (MDI), hexamethylene diisocyanate (HDI), toluene diisocyanate (TDI), isophorone diisocyanate (IPDI), metaxylylene diisocyanate (XDI), norbornane diisocyanate (NBDI), and 1,4-bis (isocyanatomethyl) cyclohexane (1,4-H6XDI).

In addition, from the viewpoint of low reactivity and a long pot life during the preparation of a reaction cured product, an aliphatic polyisocyanate compound is preferable, hexamethylene diisocyanate (HDI), isophorone diisocyanate (IPDI), metaxylylene diisocyanate (XDI), norbornane diisocyanate (NBDI), or 1,4-bis(isocyanatomethyl)cyclohexane (1,4-H6XDI) is more preferable, and metaxylylene diisocyanate (XDI) or norbornane diisocyanate (NBDI) is still more preferable.

Further, from the viewpoint of further improving the ultrasound attenuation properties, it is preferable that the polyisocyanate compound includes an aliphatic polyisocyanate compound having an aromatic ring and an aromatic polyisocyanate compound.

The isocyanate group equivalent of the polyisocyanate compound is not particularly limited and is, for example, preferably 50 to 500 g/mol and more preferably 50 to 250 g/mol. The isocyanato group equivalent means the mass (g) of the polyisocyanate compound per 1 mol of isocyanato groups.

The polyisocyanate compound may be a solid or a liquid at 25° C. In a case where the polyisocyanate compound is a liquid at 25° C., the viscosity of the polyisocyanate compound at 25° C. is not particularly limited and is, for example, preferably 1 to 30000 mPa·sec, more preferably 3 to 30000 mPa·sec, and still more preferably 3 to 20000 mPa·sec.

### (Polyamine Compound)

A polyamine compound that is reacted with the epoxy resin having a polyurethane structure in (A) can be given as an example of the polyamine compound for obtaining the reaction cured product (C), and the preferred range of the polyamine compound can be the same as described above unless otherwise specified.

Among the polyamine compounds that are reacted with the epoxy resin having a polyurethane structure in (A), an aromatic polyamine compound having a polyether structure, such as ELASTMER 250P, ELASTMER 650P, ELASTMER 1000P, or POLEA SL-100A (all of which are product names, manufactured by Kumiai Chemical Co., Ltd.), is preferable as the polyamine compound from the viewpoint of low reactivity and a long pot life during the preparation of a reaction cured product.

The content of any one of the reaction cured products (A) to (C) in the resin contained in the backing material according to the present invention is not particularly limited as long as the effects of the present invention are obtained. For example, the content can be set to 15% by volume or more, is preferably 20% by volume or more, more preferably 30% by volume or more, still more preferably 50% by volume or more, and particularly preferably 70% by volume or more. It is also preferable that all of the resins contained in the backing material according to the present invention are composed of any one of (A) to (C).

The content of the resin in the backing material is 25% to 50% by volume and is preferably 30% to 50% by volume.

### <Thermally Conductive Particles>

Any of inorganic particles or organic particles may be used as the thermally conductive particles as long as the particles have thermal conductivity (preferably, a thermal conductivity of 30 W/m·K or more). Thermally conductive particles for imparting heat dissipation properties to the backing material can be used without any particular limitation. The shape of the particles is not particularly limited, and particles having various shapes, such as an amorphous shape, a spherical shape, a fibrous shape, a branched fibrous shape, and a flat plate shape, are used. In a case where the shape is a spherical shape, it is possible to increase a filling rate, which is preferable. On the other hand, in a case where the shape is an anisotropic shape, such as a fibrous shape or a flat plate shape, it is possible to increase a particle contact and to improve heat dissipation properties, which is preferable. In addition, in the case of the amorphous particles, it is possible to randomly reflect ultrasonic waves, which is preferable from the viewpoint of improving the ultrasound attenuation properties of the backing material.

Metal particles consisting of metal materials, such as silver, copper, gold, aluminum, iron, brass, tungsten, molybdenum, and zinc, can be given as examples of the inorganic particles. Among these metal particles, tungsten or molybdenum particles are preferable from the viewpoint of low electrical conductivity.

In addition, an oxide, a carbide, or a nitride of the metal particles can also be preferably used. For example, aluminum trioxide (alumina or sapphire), zinc oxide, aluminum nitride, tungsten carbide, or molybdenum carbide is preferable.

Further, inorganic particles, such as silicon carbide and boron nitride, can also be preferably used.

That is, metal particles, ceramic particles, and the like can be preferably used as the inorganic particles.

Graphite, carbon nanotubes, and diamond can be given as examples of the organic particles. Among these metal particles, diamond is preferable from the viewpoint of low electrical conductivity.

The thermal conductivity of the thermally conductive particles is preferably 30 W/m·K or more.

A surface of the metal particle may be subjected to a surface treatment. An ultraviolet (UV) surface treatment, a plasma surface treatment, a corona surface treatment, a silane coupling treatment, a titanium coupling treatment, an aluminum coupling treatment, and a phosphoric acid treatment can be given as examples of the surface treatment. The surface treatment makes it possible to change the functional groups on the surface of the particle and to improve the dispersibility of the particles in the resin. The thermal conductivity and the ultrasound attenuation properties can be improved by changing the interparticle distance.

It is preferable that the thermally conductive particles include at least one of metal particles or ceramic particles.

The particle diameter of the thermally conductive particle is not particularly limited. The particle diameter of the thermally conductive particles is, for example, preferably 1 to 300 um, more preferably 5 to 100 μm, and still more preferably 8 to 30 μm from the viewpoint of maintaining the mechanical strength of the backing material (cured substance) according to the present invention at a high level while suppressing the viscosity of the curable resin composition, which will be described below, to a low level.

The “particle diameter” of the thermally conductive particles is a number-average particle diameter and is a value measured by a method described in Examples which will be described below.

The proportion of the thermally conductive particles to the total amount of components other than the above-described resin in the backing material is preferably 50% by volume or more, more preferably 60% by volume or more, and still more preferably 65% by volume or more. It is also preferable that all of the components other than the above-described resin in the backing material according to the present invention are thermally conductive particles.

For example, the content of the thermally conductive particles in the backing material is preferably 30% to 60% by volume, more preferably 30% to 55% by volume, and still more preferably 30% to 50% by volume.

In addition, for the thermally conductive particles, one type of thermally conductive particles may be used alone, or two or more types of thermally conductive particles may be used in combination. In the present invention, in a case where two or more types of thermally conductive particles are contained, the description of the proportion and content of the thermally conductive particles means the total amount of thermally conductive particles.

### <Other Components>

The backing material according to the present invention may contain other components in addition to the above-described resin and thermally conductive particles.

It is preferable that the other components include hollow particles. The inclusion of the hollow particles makes it possible to further improve the ultrasound attenuation properties. As the hollow particles, hollow particles commonly used for exhibiting the effect of improving acoustic attenuation properties or ultrasound attenuation properties can be used without particular limitation. Any of hollow glass particles or hollow resin particles may be used as the hollow particles, and it is preferable to use the hollow resin particles.

Preferred examples of the hollow particles include glass balloons, hollow silica, cenolite, plastic microballoons, such as phenolic resin microballoons, urea resin microballoons, and polymethyl methacrylate balloons, and thermally expandable microcapsules.

In addition, plastic microballoons whose surfaces have been coated with an inert inorganic powder, such as calcium carbonate, may be used as the hollow particles. Examples of the plastic microballoons include MFL-81GCA, MFL-SEVEN, MFL-HD30CA, MFL-HD60CA, and MFL-100MCA (all of which are product names, manufactured by Matsumoto Yushi-Seiyaku Co., Ltd.), which belong to the Matsumoto Microsphere Series.

In addition, for the hollow particles, one type of hollow particles may be used alone, or two or more types of hollow particles may be used in combination. In the present invention, in a case where two or more types of hollow particles are contained, the content of the hollow particles means the total amount of hollow particles.

The particle diameter of the hollow particles is not particularly limited. The particle diameter of the hollow particles is, for example, preferably 1 to 300 μm, more preferably 5 to 100 μm, and still more preferably 20 to 80 μm from the viewpoint of maintaining the mechanical strength of the backing material (cured product) according to the present invention at a high level while suppressing the viscosity of the curable resin composition, which will be described below, to a low level.

The “particle diameter” of the hollow particles is synonymous with the “particle diameter” of the above-described thermally conductive particles. That is, the “particle diameter” of the hollow particles is a number-average particle diameter and is a value measured by a method described in Examples which will be described below.

The other components may include a dispersant, a diluent, a colorant, a viscosity modifier, a plasticizer, a curing accelerator, and the like.

The content of the other components in the backing material can be, for example, 10% to 20% by volume.

An example of the preferred aspect of the backing material according to the present invention is a backing material for an ultrasound probe that contains a resin, which contains any one of (A) to (C) and has the above-described specific viscoelastic properties, and thermally conductive particles, has a specific storage elastic modulus, and contains the hollow particles.

In this aspect, the content of each component in the backing material is as follows: the content of the resin is 25% to 50% by volume and is preferably 30% to 50% by volume; the content of the thermally conductive particles is preferably 30% to 60% by volume, more preferably 30% to 55% by volume, and still more preferably 30% to 50% by volume; and the content of the hollow particles is preferably 10% to 20% by volume.

### <Curable Resin Composition>

The backing material according to the present invention is preferably formed using the curable resin composition according to the present invention.

The curable resin composition according to the present invention is for forming the backing material according to the present invention and contains thermally conductive particles and any one of the following (a) to (c) as a resin component: (a) a combination of an epoxy resin having a polyurethane structure and a polyamine compound; (b) a combination of an epoxy resin and a polyamine compound, at least one of the epoxy resin or the polyamine compound having a polyether structure; and (c) a combination of a polyisocyanate compound and a polyamine compound. The viscosity of the curable resin composition is 5000 Pa·sec or less under conditions of 25° C. and a shear rate of 0.01/sec.

The resin component and (a) to (c) in the curable resin composition according to the present invention correspond to the resin and (A) to (C) in the backing material according to the present invention, respectively, in a case where the backing material according to the present invention is formed.

Therefore, the description of the epoxy resin having a polyurethane structure and the polyamine compound in (A) can be applied to the epoxy resin having a polyurethane structure and the polyamine compound in (a). The description of the epoxy resin and the polyamine compound in (B) can be applied to the epoxy resin and the polyamine compound in (b). The description of the polyisocyanate compound and the polyamine compound in (C) can be applied to the polyisocyanate compound and the polyamine compound in (c).

In addition, for the combination of an epoxy resin having a polyurethane structure and a polyamine compound, in a case where at least one of the epoxy resin having a polyurethane structure or the polyamine compound has a polyether structure, the combination is classified as (a) instead of (b). Therefore, the epoxy resin in (b) does not have a polyurethane structure.

Further, for the combination of a polyisocyanate compound and a polyamine compound, in a case where at least one of the polyisocyanate compound or the polyamine compound has a polyether structure, the combination is classified as (c) instead of (b).

Furthermore, the mixing ratio of the epoxy resin having a polyurethane structure and the polyamine compound in (a) can be appropriately adjusted such that, in the obtained backing material according to the present invention, the resin including (A) has specific viscoelastic properties and the backing material has a specific storage elastic modulus. The same applies to (b) and (c). That is, the mixing ratio of the epoxy resin and the polyamine compound (however, at least one of the epoxy resin or the polyamine compound has a polyether structure) in (b) can be appropriately adjusted such that, in the obtained backing material according to the present invention, the resin including (B) has specific viscoelastic properties and the backing material has a specific storage elastic modulus. The mixing ratio of the polyisocyanate compound and the polyamine compound in (c) can be appropriately adjusted such that, in the obtained backing material according to the present invention, the resin including (C) has specific viscoelastic properties and the backing material has a specific storage elastic modulus.

The thermally conductive particles in the curable resin composition according to the present invention correspond to the thermally conductive particles in the backing material according to the present invention. Therefore, the description of the thermally conductive particles in the backing material according to the present invention can be applied to the thermally conductive particles in the curable resin composition according to the present invention.

In addition, the curable resin composition according to the present invention can also contain the other components described in the backing material according to the present invention.

### (Viscosity of Curable Resin Composition)

The viscosity of the curable resin composition according to the present invention is 5000 Pa·sec or less under conditions of 25° C. and a shear rate of 0.01/sec.

The viscosity of the curable resin composition according to the present invention is measured by a method described in Examples which will be described below.

In a case where the curable resin composition according to the present invention satisfies the specific viscosity (viscosity), the curable resin composition has sufficient fluidity to be poured into a mold and can be molded into a desired shape.

The viscosity of the curable resin composition according to the present invention under the conditions of 25° C. and a shear rate of 0.01/sec is preferably 3000 Pa·sec or less, more preferably 2000 Pa·sec or less, still more preferably 1000 Pa·sec or less, and particularly preferably 800 Pa·sec or less.

The content of the thermally conductive particles and the resin component in the curable resin composition according to the present invention is not particularly limited as long as the backing material according to the present invention can be obtained.

The content of any one of (a) to (c) in the resin component of the curable resin composition according to the present invention is not particularly limited as long as the effects of the present invention are obtained. For example, the content may be 15% by volume or more, is preferably 20% by volume or more, more preferably 30% by volume or more, still more preferably 50% by volume or more, and particularly preferably 70% by volume or more. The upper limit value of the content is not particularly limited and can be set to 100% by volume or less.

The content of the resin component in the curable resin composition according to the present invention is preferably 25% to 50% by volume and more preferably 30% to 50% by volume.

The proportion of the thermally conductive particles to the total amount of components other than the above-described resin component in the curable resin composition according to the present invention is preferably 50% by volume or more, more preferably 60% by volume or more, and still more preferably 70% by volume or more. It is also preferable that all of the components other than the above-described resin component in the curable resin composition according to the present invention are thermally conductive particles.

The content of the thermally conductive particles in the curable resin composition according to the present invention is, for example, preferably 30% to 60% by volume, more preferably 30% to 55% by volume, and still more preferably 30% to 50% by volume.

In addition, in a case where the curable resin composition according to the present invention contains the other components described above, the content of the other components in the curable resin composition according to the present invention can be set to, for example, 10% to 20% by volume.

An example of the preferred aspect of the curable resin composition according to the present invention is a curable resin composition that contains a resin component containing any one of (a) to (c) and thermally conductive particles, has the above-described specific viscoelastic properties, and contains the hollow particles.

In this aspect, the content of each component in the curable resin composition is as follows: the content of the resin component is preferably 25% to 50% by volume and more preferably 30% to 50% by volume; the content of the thermally conductive particles is preferably 30% to 60% by volume, more preferably 30% to 55% by volume, and still more preferably 30% to 50% by volume; and the content of the hollow particles is preferably 10% to 20% by volume.

### <Method for Manufacturing Backing Material>

The curable resin composition according to the present invention can be prepared by a conventional method.

For example, as the components constituting the curable resin composition according to the present invention, the above-described thermally conductive particles, the above-described resin component containing any one of (a) to (c), and other appropriate components can be kneaded together by a planetary device, such as a planetary mixer, and a kneading device, such as a kneader, a pressure kneader, a Banbury mixer (continuous kneader), or a two-roll mill, to obtain the curable resin composition. A mixing order of each component is not particularly limited. Kneading conditions are not particularly limited. The thermally conductive particles and other components that may be appropriately contained may be dispersed in the resin component.

The curable resin composition according to the present invention obtained in this way can be cured to obtain the backing material according to the present invention. Curing conditions can be adjusted according to the chemical reaction of the resin component contained in the curable resin composition according to the present invention. For example, the curable resin composition can be heated and cured at 20° C. to 200° C. for 5 to 500 minutes to obtain the backing material.

The shape of the backing material is not particularly limited. For example, the backing material may be formed in a preferred shape as a backing member by a mold during curing. Alternatively, a desired backing member may be obtained by preparing a sheet-shaped backing material and cutting the sheet-shaped backing material by dicing or the like.

Further, since the backing material according to the embodiment of the present disclosure has excellent workability, it is possible to manufacture a desired backing member while suppressing the occurrence of deformation, breakage, and the like even in a case where the backing material is diced into a desired shape at a pitch on the order of micrometers.

The backing material according to the present invention is useful for medical members. For example, the backing material can be preferably used for an acoustic probe and an acoustic measurement device and can be more preferably used for an ultrasound probe and an ultrasound diagnostic apparatus. In addition, the acoustic measurement device according to the present invention is not limited to an ultrasound diagnostic apparatus or a photoacoustic measurement device and refers to a device that receives acoustic waves reflected from or generated by an object and displays the received acoustic waves as an image or signal intensity.

In particular, the backing material according to the present invention can be suitably used as, for example, a backing member for an ultrasound probe, a material of a backing member in a photoacoustic measurement device or an ultrasound endoscope, and a material of a backing member in an ultrasound probe comprising capacitive micromachined ultrasonic transducers (cMUT) as an ultrasound transducer array.

Specifically, the backing material according to the present invention is preferably applied to acoustic measurement devices, such as the ultrasound diagnostic apparatuses described in JP2003-169802A and the like and the photoacoustic measurement devices described in JP2013-202050A, JP2013-188465A, and the like.

### <<Acoustic Probe>>

A configuration of the acoustic probe using the backing material according to the present invention will be described in detail below based on the configuration of the ultrasound probe in the ultrasound diagnostic apparatus shown in the FIGURE. In addition, the ultrasound probe is a probe that particularly uses ultrasonic waves as the acoustic waves of the acoustic probe. Therefore, a basic structure of the ultrasound probe can be applied to the acoustic probe without any change.

### Ultrasound Probe

The configuration of the ultrasound probe according to the present invention will be described in more detail below based on the configuration of the ultrasound probe in the ultrasound diagnostic apparatus shown in the FIGURE.

An ultrasound probe 10 is a main component of the ultrasound diagnostic apparatus and has a function of generating ultrasonic waves and transmitting and receiving ultrasonic beams. As shown in the FIGURE, the ultrasound probe 10 is configured such that an acoustic lens 1, an acoustic matching layer 2, a piezoelectric element layer 3, and a backing member 4 are provided in order from a distal end portion (a surface coming into contact with a living body which is a test object). Further, in recent years, an ultrasound probe having a laminated structure of an ultrasound transducer (piezoelectric element) for transmission and an ultrasound transducer (piezoelectric element) for reception that are made of different materials has been proposed in order to receive high-order harmonics.

### <Piezoelectric Element Layer>

The piezoelectric element layer 3 is a portion which generates ultrasonic waves and in which electrodes are attached to both sides of a piezoelectric element. In a case where a voltage is applied, the piezoelectric element repeatedly expands and contracts and vibrates to generate ultrasonic waves.

A so-called ceramics inorganic piezoelectric body obtained by performing a polarization treatment on quartz crystals, single crystals, such as LiNbO₃, LiTaO₃, and KNbO₃, thin films, such as ZnO and AlN thin films, sintered bodies, such as Pb(Zr, Ti)O₃-based sintered bodies, is widely used as the material constituting the piezoelectric element. In general, piezoelectric ceramics, such as lead zirconate titanate (PZT) with good conversion efficiency, are used.

In addition, a piezoelectric element that detects received waves on the higher frequency side requires sensitivity over a wider bandwidth. For this reason, an organic piezoelectric body using an organic polymer material, such as polyvinylidene fluoride (PVDF), is used as the piezoelectric element that is suitable for a high frequency or a wide band.

Furthermore, cMUT using micro electro mechanical systems (MEMS) technology, in which an array structure having excellent short pulse characteristics, excellent broadband characteristics, excellent mass productivity, and a minimal variation in characteristics is obtained, is described in JP2011-071842A or the like.

In the present invention, it is possible to preferably use any piezoelectric element material.

### <Backing Member>

The backing member 4 is provided on a rear surface of the piezoelectric element layer 3, suppresses excess vibrations to reduce the pulse width of the ultrasonic waves, and contributes to improving the distance resolution in an ultrasound diagnostic image.

In the ultrasound probe according to the present invention, the backing member 4 includes the backing material according to the present invention. The backing material according to the present invention has excellent ultrasound attenuation properties and excellent workability and also has high heat dissipation properties achieved by the thermally conductive particles. Therefore, the use of the backing material according to the present invention or the curable resin composition according to the present invention makes it possible to obtain the backing member 4, which can suppress excess vibrations radiated to the rear surface from the piezoelectric element layer 3 during the driving of the ultrasound probe and efficiently dissipate the heat generated from the piezoelectric element layer 3, with high productivity, while suppress the occurrence of deformation, breakage, and the like.

### <Acoustic Matching Layer>

The acoustic matching layer 2 is provided in order to reduce the difference in acoustic impedance between the piezoelectric element layer 3 and the test object and to efficiently transmit and receive the ultrasonic waves.

### <Acoustic Lens>

The acoustic lens 1 is provided to focus the ultrasonic waves in a slice direction, using refraction, to improve the resolution. In addition, the acoustic lens 1 is required to come into close contact with the living body, which is the test object, to match the ultrasonic waves with the acoustic impedance (1.4 to 1.7 Mrayl in a case of a human body) of the living body.

That is, the transmission and reception sensitivity of the ultrasonic waves is improved using a material, whose sound speed is sufficiently lower than the sound speed of the human body and whose acoustic impedance is close to the value of the skin of the human body, as the material forming the acoustic lens 1.

The operation of the ultrasound probe 10 having this configuration will be described. A voltage is applied to the electrodes provided on both sides of the piezoelectric element layer 3 to resonate the piezoelectric element layer 3 such that an ultrasound signal is transmitted from the acoustic lens 1 to the test object. During the reception of the ultrasound signal, the piezoelectric element layer 3 is vibrated by a signal (echo signal) reflected from the test object, and this vibration is electrically converted into a signal to obtain an image.

Ultrasound Probe Comprising Capacitive Micromachined Ultrasonic Transducer (cMUT)

In a case where the cMUT device described in JP2006-157320A, JP2011-71842A, or the like is used in the ultrasound transducer array, the sensitivity of the cMUT device is generally lower than the sensitivity of a transducer using general piezoelectric ceramics (PZT).

In addition, since the cMUT device is manufactured by MEMS technology, it is possible to provide the market with an ultrasound probe having higher mass productivity and lower cost than a piezoelectric ceramic probe.

### Photoacoustic Measurement Device Using Photoacoustic Imaging

Photoacoustic imaging (PAI) described in JP2013-158435A or the like displays an image or signal intensity of the ultrasonic waves generated in a case where the inside of a human body is irradiated with light (electromagnetic waves) and the human tissues adiabatically expand due to the irradiated light.

### Ultrasound Endoscope

The ultrasound endoscope described in JP2008-311700A or the like comprise an insertion portion that is inserted into a body and an operation portion that is connected to a base end of the insertion portion. An ultrasound probe is provided at a distal end of the insertion portion.

In a case where a backing member including the backing material according to the present invention is used as the backing member constituting the ultrasound probe, excess vibrations applied to the ultrasound endoscope are suppressed. Therefore, the acoustic characteristics of the ultrasound endoscope are improved.

### EXAMPLES

Hereinafter, the present invention will be described in more detail through Examples, but the present invention should not be construed as being limited thereto.

### Examples

### <1> Preparation of Backing Member Composition

Backing member compositions (curable resin compositions) having the compositions shown in Tables 1-1 and 1-2 (hereinafter, collectively referred to as “Table 1”) were prepared.

Specifically, resin raw materials, thermally conductive particles (WC-3 and SiC-1), and hollow particles (B-1) were weighed according to the content ratios shown in Table 1 and mixed by a planetary mixer (product name: Awatori Rentaro Vacuum Type ARV-310, manufactured by Thinky Corporation) to prepare backing member composition Nos. 101 to 125 and c01 to c18.

### <2> Production of Backing Member Sheet

The backing member composition prepared as described above was poured into a square mold having one side of 30 mm and a desired depth, heated at 80° C. for 18 hours, and then heated at 150° C. for 1 hour to cure, thereby producing a square backing member sheet having one side of 30 mm and a desired thickness. Then, the backing member sheet was used for the following measurement and evaluation. Backing member sheet Nos. 101 to 125 are the backing materials according to the present invention, and backing member sheet Nos. c01 to c18 are comparative backing materials.

The depth of the mold used and the thickness of the obtained sheet are 2 mm or 0.5 mm, respectively.

### <3> Measurement and Evaluation

The following measurements and evaluations were performed on the above-described backing member compositions and the above-described backing member sheets. The results are summarized in Table 1.

### (1) Measurement of Storage Elastic Modulus

The square backing member sheet having a thickness of 0.5 mm was cut into a strip shape having a width of 5 mm to prepare a test piece. The storage elastic modulus of the produced test piece was measured under conditions of a grip distance of 20 mm, a temperature rise rate of 2° C./min, a measurement temperature range of −150° C. to 250° C., and a frequency of 5 Hz, using a dynamic viscoelasticity measurement device itk DVA-225 (product name, manufactured by IT Keisoku & Control Co., Ltd.).

The minimum value of the storage elastic modulus in a range of 0° C. to 50° C. (hereinafter, also abbreviated as “elastic modulus”) was calculated and evaluated based on the following criteria.

### Evaluation Criteria (Elastic Modulus)

S: The elastic modulus is 3300 MPa or more and less than 8000 MPa.
A: The elastic modulus is 3000 MPa or more and less than 3300 MPa.
B: The elastic modulus is 2500 MPa or more and less than 3000 MPa.
C: The elastic modulus is 2000 MPa or more and less than 2500 MPa.
D: The elastic modulus is 1000 MPa or more and less than 2000 MPa.
E: The elastic modulus is less than 1000 MPa.

### (2) Attenuation Rate

The intensity of a reflected echo was measured using a sing-around-type sound speed measurement device (manufactured by Ultrasonic Engineering Co., Ltd., product name “Ultrasonic Velocity Measurement Device UVM-2 Type”) based on the method described in the method for measuring an ultrasound attenuation coefficient of solids according to JIS (Japanese Industrial Standards) Z 2354 (2012). In addition, in the measurement, a measurement probe of 2 MHz was used in water at 25° C., a backing member sheet having a thickness of 2 mm was used as a test piece for measurement, and an attenuation rate was calculated from a difference in the intensity of the reflected echo depending on the presence or absence of the test piece for measurement and the thickness of the test piece for measurement. Then, the evaluation was performed based on the following criteria.

### Evaluation Criteria (Attenuation Rate)

S: The attenuation rate is more than 4.0 dB/(mm·MHz).
A: The attenuation rate is more than 3.5 dB/(mm·MHz) and is 4.0 dB/(mm·MHz) or less.
B: The attenuation rate is more than 3.0 dB/(mm·MHz) and is 3.5 dB/(mm·MHz) or less.
C: The attenuation rate is more than 2.5 dB/(mm·MHz) and is 3.0 dB/(mm·MHz) or less.
D: The attenuation rate is more than 0.8 dB/(mm·MHz) and is 2.5 dB/(mm·MHz) or less.
E: The attenuation rate is 0.8 dB/(mm·MHz) or less.

### (3) Viscosity

The viscosity of the backing member composition was measured using a HAAKE MARS 40 rheometer (product name, manufactured by Thermo Fisher Scientific Inc.) under the following conditions: temperature: 25° C.; shear rate: 0.01/sec; sensor: C35 2°/Ti; measurement mode: oscillation mode; and frequency: 0.03 Hz. Then, the viscosity was evaluated based on the following criteria.

### Evaluation Criteria (Viscosity)

S: The viscosity is 800 Pa·sec or less.
A: The viscosity is more than 800 Pa·sec and is 1000 Pa·sec or less.
B: The viscosity is more than 1000 Pa·sec and is 2000 Pa·sec or less.
C: The viscosity is more than 2000 Pa·sec and is 3000 Pa·sec or less.
D: The viscosity is more than 3000 Pa·sec and is 5000 Pa·sec or less.
E: The viscosity is more than 5000 Pa·sec.

### (4) Workability

Cross-dicing was performed on the backing member sheet having a thickness of 0.5 mm, using an automatic dicing saw DAD321 (product name, manufactured by DISCO Corporation) and a diamond blade having a thickness of 0.03 mm, to form grooves having a width of 30 μm and a depth of 200 μm, and 20 rows×20 columns of squares (a total of 400 squares) each having a size of 200 μm×200 μm were formed. The squares formed by the cross dicing were observed with a microscope, the number of damaged and/or collapsed squares among the 400 squares was counted, and the workability was evaluated based on the following criteria.

### Evaluation Criteria (Workability)

S: There are no damaged and/or collapsed squares.
A: The number of damaged and/or collapsed squares is one or more and four or less.
B: The number of damaged and/or collapsed squares is five or more and 20 or less.
C: The number of damaged and/or collapsed squares is 21 or more and 40 or less.
D: The number of damaged and/or collapsed squares is 41 or more and 80 or less.
E: The number of damaged and/or collapsed squares is 81 or more.

TABLE 1

| No. | | 101 | 102 | 103 | 104 | 105 | 106 | 107 | 108 | 109 | 110 | 111 | 112 | 113 | 114 | 115 |
|---|---|---|---|---|---|---|---|---|---|---|---|---|---|---|---|---|
| Resin raw | Type | R-1 | R-2 | R-3 | R-4 | R-5 | R-6 | R-7 | R-8 | R-9 | R-10 | R-11 | R-12 | R-13 | R-14 | R-15 |
| material | Content | 45 | 45 | 45 | 45 | 45 | 45 | 45 | 45 | 45 | 45 | 45 | 45 | 45 | 45 | 45 |
| WC-3 | Content | 25 | 25 | 25 | 25 | 25 | 25 | 25 | 25 | 25 | 25 | 25 | 25 | 25 | 25 | 25 |
| SiC-1 | Content | 15 | 15 | 15 | 15 | 15 | 15 | 15 | 15 | 15 | 15 | 15 | 15 | 15 | 15 | 15 |
| B-1 | Content | 15 | 15 | 15 | 15 | 15 | 15 | 15 | 15 | 15 | 15 | 15 | 15 | 15 | 15 | 15 |
| Tanδmin (0~50° C.) | | 0.06 | 0.07 | 0.08 | 0.07 | 0.10 | 0.06 | 0.37 | 0.13 | 0.14 | 0.13 | 0.07 | 0.08 | 0.08 | 0.06 | 0.06 |
| Tanδmax (−20~110° C.) | | 0.80 | 0.90 | 0.80 | 0.50 | 0.45 | 0.90 | 1.40 | 1.40 | 1.40 | 0.95 | 0.63 | 0.15 | 0.15 | 0.13 | 0.12 |
| E′ (0~50° C.) | | A | A | A | A | A | A | C | C | C | C | C | S | A | C | A |
| Evaluation | Attenuation rate | C | C | A | S | S | C | A | A | A | S | A | A | S | A | A |
| | Viscosity | B | B | A | A | A | B | B | A | A | S | S | C | A | A | B |
| | Workability | C | C | C | B | B | D | D | D | D | D | C | A | A | A | A |

| No. | | 116 | 117 | 118 | 119 | 120 | 121 | 122 | 123 | 124 | 125 |
|---|---|---|---|---|---|---|---|---|---|---|---|
| Resin raw | Type | R-16 | R-17 | R-18 | R-19 | R-19 | R-19 | R-20 | R-21 | R-22 | R-23 |
| material | Content | 45 | 45 | 45 | 45 | 25 | 50 | 45 | 45 | 45 | 45 |
| WC-3 | Content | 25 | 25 | 25 | 25 | 35 | 20 | 25 | 25 | 25 | 25 |
| SiC-1 | Content | 15 | 15 | 15 | 15 | 25 | 15 | 15 | 15 | 15 | 15 |
| B-1 | Content | 15 | 15 | 15 | 15 | 15 | 15 | 15 | 15 | 15 | 15 |
| Tanδmin (0~50° C.) | | 0.08 | 0.08 | 0.08 | 0.08 | 0.08 | 0.08 | 0.08 | 0.08 | 0.08 | 0.08 |
| Tanδmax (−20~110° C.) | | 0.18 | 0.15 | 0.15 | 0.20 | 0.20 | 0.20 | 0.20 | 0.15 | 0.15 | 0.15 |
| E′ (0~50° C.) | | A | A | B | S | S | B | A | A | A | A |
| Evaluation | Attenuation rate | S | B | B | S | A | A | S | B | B | B |
| | Viscosity | A | B | B | A | D | A | A | D | D | D |
| | Workability | A | A | A | A | A | A | A | A | A | A |

| No. | | c01 | c02 | c03 | c04 | c05 | c06 | c07 | c08 | c09 | c10 |
|---|---|---|---|---|---|---|---|---|---|---|---|
| Resin raw | Type | PR-1 | PR-2 | PR-3 | PR-4 | PR-5 | PR-6 | PR-7 | PR-8 | PR-9 | PR-10 |
| material | Content | 45 | 45 | 45 | 45 | 45 | 45 | 45 | 45 | 45 | 45 |
| WC-3 | Content | 25 | 25 | 25 | 25 | 25 | 25 | 25 | 25 | 25 | 25 |
| SiC-1 | Content | 15 | 15 | 15 | 15 | 15 | 15 | 15 | 15 | 15 | 15 |
| B-1 | Content | 15 | 15 | 15 | 15 | 15 | 15 | 15 | 15 | 15 | 15 |
| Tanδmin (0~50° C.) | | 0.02 | 0.03 | 0.03 | 0.02 | 0.05 | 0.07 | 0.06 | 0.02 | 0.02 | 0.02 |
| Tanδmax (−20~110° C.) | | 1.10 | 1.10 | 1.10 | 1.52 | 0.60 | 1.54 | 1.21 | 1.10 | 1.10 | 1.10 |
| E′ (0~50° C.) | | A | A | A | A | A | D | E | A | A | A |
| Evaluation | Attenuation rate | E | E | E | E | E | A | A | E | E | E |
| | Viscosity | E | E | E | A | D | A | A | E | E | E |
| | Workability | D | D | D | E | B | E | E | D | D | D |

| No. | | c11 | c12 | c13 | c14 | c15 | c16 | c17 | c18 |
|---|---|---|---|---|---|---|---|---|---|
| Resin raw | Type | S-1 | SE-1 | RB-1 | EV-1 | NBR-1 | IR-1 | R-19 | R-19 |
| material | Content | 45 | 45 | 45 | 45 | 45 | 45 | 20 | 55 |
| WC-3 | Content | 25 | 25 | 25 | 25 | 25 | 25 | 35 | 20 |
| SiC-1 | Content | 15 | 15 | 15 | 15 | 15 | 15 | 25 | 15 |
| B-1 | Content | 15 | 15 | 15 | 15 | 15 | 15 | 20 | 10 |
| Tanδmin (0~50° C.) | | 0.05 | 0.05 | 0.30 | 0.30 | 0.05 | 0.05 | 0.08 | 0.08 |
| Tanδmax (−20~110° C.) | | 0.40 | 0.40 | 0.40 | 0.43 | 0.30 | 0.30 | 0.21 | 0.20 |
| E′ (0~50° C.) | | E | E | E | E | E | E | S | E |
| Evaluation | Attenuation rate | A | A | A | A | A | A | A | A |
| | Viscosity | E | E | E | E | E | E | E | A |
| | Workability | E | E | E | E | E | E | E | E |

<Note in Table>
(Resin Raw Material)
R-1 to R-23 and PR-1 to PR-10: R-1 to R-23 and PR-1 to PR-10 described in the following Tables A-1 to A-3 (hereinafter, collectively referred to as “Table A”), which are obtained by mixing a main agent and a curing agent at the mixing ratios described in Table A.
S-1, SE-1, RB-1, EV-1, NBR-1, and IR-1: S-1, SE-1, RB-1, EV-1, NBR-1, and IR-1 shown in the following Table C
(Thermally Conductive Particles)
WC-3: tungsten carbide particles, WC100S (product name), manufactured by A.L.M.T. Corp., particle diameter: 10 μm
SiC-1: silicon carbide particles, SSC-A15 (product name), manufactured by Shinano Electric Refining Co., Ltd., particle diameter: 17 μm
(Hollow Particles)
B-1: hollow resin particles, Matsumoto Microsphere MFL-HD60CA (product name), manufactured by Matsumoto Yushi-Seiyaku Co., Ltd., plastic microballoons whose surfaces were coated with calcium carbonate powder, the values of the particle diameters of all sheets were in a range of 50 to 70 μm, which differed depending on the type of the backing member sheet (Nos. 101 to 125 and c01 to c18).

In addition, the particle diameters of the thermally conductive particles and the hollow particles are number-average particle diameters measured by the following method. Further, for the hollow particles, the presence of the hollow particles in the backing member sheet was confirmed by cutting the backing member sheet with a razor and observing the cross section with a tabletop microscope Miniscope TM4000 (product name, manufactured by Hitachi High-Tech Corporation).

### (Measurement of Number-Average Particle Diameter of Particles)

The number-average particle diameter of the particles was calculated by observing the end surface of the backing member sheet with a scanning electron microscope (SU8030 (product name) manufactured by Hitachi High-Tech Corporation) in a visual field including 500 or more particles, randomly extracting 500 particles from the particles in the visual field, and measuring the particle diameters of the extracted particles.

The backing member sheet, whose end surface could not be observed with the scanning electron microscope, was observed by ice-embedded transmission electron microscopy. The ice embedding was performed using Vitrobot Mark IV (product name) manufactured by FEI Company, 500 particles were randomly extracted from the particles in the visual field with a transmission electron microscope (JEM-2010 (product name) manufactured by JEOL Ltd.), and the particle diameters of the extracted particles were measured and calculated.

In a case where the particles were not perfectly circular, particles with a maximum major to minor axis ratio of 0.7 or more were extracted and measured.

The contents of the resin raw material, the thermally conductive particles (WC-3 and SiC-1), and the hollow particles (B-1) are content ratios based on the volume.

tan δ min (0° C. to 50° C.): the minimum value of the loss tangent of the resin in a range of 0° C. to 50° C. which was measured by the following method.

tan δ max (−20° C. to 110° C.): the maximum value of the loss tangent of the resin in a range of −20° C. to 110° C. which was measured by the following method.

E′ (0° C. to 50° C.): the evaluation result related to the minimum value of the storage elastic modulus of the backing member sheet in a range of 0° C. to 50° C. which was measured and evaluated as described above.

### [Measurement of Loss Tangent of Resin]

### (1) Production of Resin Sheet

For the resin raw materials R-1 to R-23 and PR-1 to PR-10, the main agent and the curing agent shown in Table A were weighed according to the mixing ratio shown in Table A, mixed with a planetary mixer (product name: Awatori Nentaro Vacuum Type ARV-310, manufactured by Thinky Corporation), molded into a sheet shape having a length of 30 mm, a width of 5 mm, and a thickness of 0.5 mm, cured at 100° C. for 2 hours, and then cured at 150° C. for 2 hours to produce a resin sheet.

For the resin raw materials S-1, SE-1, RB-1, EV-1, NBR-1, and IR-1, the resin raw materials were put into a planetary mixer (product name: Awatori Nentaro Vacuum Type ARV-310, manufactured by Thinky Corporation) as is, molded into a sheet shape having a length of 30 mm, a width of 5 mm, and a thickness of 0.5 mm, cured at 100° C. for 2 hours, and then cured at 150° C. for 2 hours to produce a resin sheet.

### (2) Evaluation of Viscoelasticity of Resin Sheet

The loss tangent of the produced resin sheet was measured under conditions of a grip distance of 20 mm, a temperature rise rate of 2° C./min, a measurement temperature range of −150° C. to 250° C., and a frequency of 5 Hz, using the dynamic viscoelasticity measurement device itk DVA-225 (product name, manufactured by IT Keisoku & Control Co., Ltd.).

TABLE A

| Resin raw material | | | | | | | | | | | | | |
|---|---|---|---|---|---|---|---|---|---|---|---|---|---|
| | | Functional group | | | | | | | | | | | |
| | | equivalent | R-1 | R-2 | R-3 | R-4 | R-5 | R-6 | R-7 | R-8 | R-9 | R-10 | R-11 |
| Main | E-1 | 189 | | | | | | | | | | 10 | 10 |
| agent | PU-1 | 230 | 10 | | | | | | | | | | |
| | PU-2 | 280 | | 10 | 10 | 10 | 10 | | | | | | |
| | PU-3 | 245 | | | | | | 10 | | | | | |
| | PEE-1 | 320 | | | | | | | 10 | 10 | | | |
| | PEE-2 | 510 | | | | | | | | | 10 | | |
| Curing | A-5 | 79 | | 1.4 | | 0.6 | 0.6 | | 1.2 | | | | |
| agent | A-7 | 110 | 2.4 | | 2.0 | 1.0 | 1.0 | 2.2 | | | 1.1 | | |
| | PEA-1 | 120 | | | | | | | | 1.9 | | | |
| | PEA-2 | 230 | | | | | | | | | | 4.5 | |
| | PEA-3 | 1028 | | | | | 0.7 | | | | | 6.0 | |
| | PEA-4 | 162 | | | | | | | | | | | 4.0 |
| | PEA-5 | 10904 | | | | | | | | | | | 4 |

| Resin raw material | | | | | | | | | | | | | | |
|---|---|---|---|---|---|---|---|---|---|---|---|---|---|---|
| | | Functional group | | | | | | | | | | | | |
| | | equivalent | R-12 | R-13 | R-14 | R-15 | R-16 | R-17 | R-18 | R-19 | R-20 | R-21 | R-22 | R-23 |
| Main | I-1 | 125 | 5.1 | 2.8 | | | | | | | 2.0 | 23 | 15.8 | 18.4 |
| agent | I-2 | 84 | | | 1.9 | | | | | | | | | |
| | I-3 | 87 | | | | 2.0 | | | | | | | | |
| | I-4 | 94 | | | | | 2.1 | | | 2.5 | | | | |
| | I-5 | 136 | | | | | | 3.1 | | | | | | |
| | I-6 | 181 | | | | | | | 4.1 | | | | | |
| Curing | A-1 | 244 | 10 | | | | | | | 2 | | | | |
| agent | A-2 | 444 | | 10 | 10 | 10 | 10 | 10 | 10 | 8 | | | | |
| | A-3 | 619 | | | | | | | | | 10 | | | |
| | A-4 | 54 | | | | | | | | | | 10 | | |
| | A-5 | 79 | | | | | | | | | | | 10 | |
| | A-6 | 68 | | | | | | | | | | | | 10 |

| Resin raw material | | | | | | | | | | | | |
|---|---|---|---|---|---|---|---|---|---|---|---|---|
| | | Functional group | | | | | | | | | | |
| | | equivalent | PR-1 | PR-2 | PR-3 | PR-4 | PR-5 | PR-6 | PR-7 | PR-8 | PR-9 | PR-10 |
| Main | E-1 | 189 | 10 | | | 10 | | 10 | 10 | | | |
| agent | E-2 | 170 | | | | | | | | 10 | | |
| | E-3 | 172 | | | | | | | | | 10 | |
| | E-4 | 175 | | | | | | | | | | 10 |
| | PU-1 | 230 | | 10 | | | | | | | | |
| | PU-3 | 245 | | | 10 | | | | | | | |
| | I-3 | 87 | | | | | 3.6 | | | | | |
| Curing | A-1 | 244 | | | | | 10 | | | | | |
| agent | A-5 | 79 | 2.1 | 3.4 | 3.2 | | | | | 2.3 | 2.3 | 2.3 |
| | PEA-1 | 120 | | | | 3.2 | | 2.2 | 2.2 | | | |
| | PEA-3 | 1028 | | | | | | 4.1 | 6.2 | | | |

TABLE B

| Main agent or | | | | Functional group | |
|---|---|---|---|---|---|
| curing agent | Type | Product name | Sales company name | equivalent | Viscosity |
| E-1 | Bisphenol A-type epoxy resin | jER 828 | Mitsubishi Chemical | 189 | 13000 |
| | | | Corporation | | |
| E-2 | Bisphenol F-type epoxy resin | jER 807 | Mitsubishi Chemical | 170 | 3500 |
| | | | Corporation | | |
| E-3 | Bisphenol E-type epoxy resin | EPOX MK R710 | Printec Corporation | 172 | 3500 |
| E-4 | Novolac-type epoxy resin | EPICLON N-730A | DIC | 175 | 40000 |
| PU-1 | Polyurethane-modified epoxy resin | ADEKA RESIN EPU-7N | ADEKA | 230 | 13000 |
| PU-2 | Polyurethane-modified epoxy resin | ADEKA RESIN EPU-11F | ADEKA | 280 | 20000 |
| PU-3 | Polyurethane-modified epoxy resin | ADEKA RESIN EPU-73B | ADEKA | 245 | 130000 |
| PEE-1 | Polyether-modified epoxy resin | ADEKA RESIN EP-4000 | ADEKA | 320 | 4500 |
| PEE-2 | Polyether-modified epoxy resin | ADEKA RESIN EP-4005 | ADEKA | 510 | 800 |
| I-1 | Polyisocyanate | Diphenylmethane | Tokyo Chemical | 125 | Solid |
| | | diisocyanate (MDI) | Industry Co., Ltd. | | |
| I-2 | Polyisocyanate | Hexamethylene | Tokyo Chemical | 84 | 4 |
| | | diisocyanate (HDI) | Industry Co., Ltd. | | |
| I-3 | Polyisocyanate | Toluene-2,4- | Tokyo Chemical | 87 | 3 |
| | | diisocyanate (TDI) | Industry Co., Ltd. | | |
| I-4 | Polyisocyanate | Metaxylylene | Tokyo Chemical | 94 | 3 |
| | | diisocyanate (XDI) | Industry Co., Ltd. | | |
| I-5 | Polymeric MDI | MILLIONATE MR-100 | Tosoh Corporation | 136 | 200 |
| I-6 | Isocyanurate-type HDI | DURANATE TPA-100 | Asahi Kasei Corporation | 181 | 1400 |
| PEA-1 | Bifunctional polyether polyamine | JEFFAMINE D230 | Huntsman Corporation | 120 | 10 |
| PEA-2 | Bifunctional polyether polyamine | JEFFAMINE D400 | Huntsman Corporation | 230 | 20 |
| PEA-3 | Bifunctional polyether polyamine | JEFFAMINE D2000 | Huntsman Corporation | 1028 | 250 |
| PEA-4 | Trifunctional polyether polyamine | JEFFAMINE T403 | Huntsman Corporation | 162 | 70 |
| PEA-5 | Trifunctional polyether polyamine | JEFFAMINE T5000 | Huntsman Corporation | 10904 | 800 |
| A-1 | Aromatic polyamine | ELASMER 250P | Kumiai Chemical Co., Ltd. | 244 | 150000 |
| A-2 | Aromatic polyamine | ELASMER 650P | Kumiai Chemical Co., Ltd. | 444 | 600 |
| A-3 | Aromatic polyamine | ELASMER 1000P | Kumiai Chemical Co., Ltd. | 619 | 5000 |
| A-4 | Aromatic polyamine | Meta-Phenylenediamine | FUJIFILM WAKO | 54 | Solid |
| A-5 | Aliphatic polyamine | 2,2,4-Trimethyl- | Tokyo Chemical | 79 | 3 |
| | | hexamethylenediamine | Industry Co., Ltd. | | |
| A-6 | Aliphatic polyamine | Metaxylylene diamine | Tokyo Chemical | 68 | 5 |
| | | | Industry Co., Ltd. | | |
| A-7 | Aliphatic polyamine | Gaskamine-328 | Mitsubishi Gas | 110 | 10000 |
| | | | Chemical Company, Inc. | | |

TABLE C

| Resin raw material | Type | Product name | Sales company name |
|---|---|---|---|
| S-1 | Silicone resin | KE-45 | Shin-Etsu Silicones |
| SE-1 | Silicone resin-modified epoxy resin | EP001K | Cemedine Co., Ltd. |
| RB-1 | EPDM (ethylene-propylene) rubber | ESPRENE 532 | Sumitomo Chemical Co., Ltd. |
| EV-1 | Ethylene-vinyl acetate copolymer resin | EVAFLEX EV-45LX | Mitsui DuPont Polychemicals Co., Ltd. |
| NBR-1 | Liquid nitrile rubber | LIQUID NBR | Zeon Corporation (Japan) |
| IR-1 | Isoprene rubber | KURAPRENE LIR | Kuraray Co., Ltd. |

### <Note in Table>

The main agent and the curing agent in Table A are as shown in Table B.

The mixing ratio of the main agent and the curing agent is a mixing ratio based on mass.

A blank cell in rows of the main agent and the curing agent in the table means that the agent is not contained.

The functional group equivalent means the mass (g) of the compound per 1 mol of functional groups (epoxy groups, isocyanate groups, or amino groups), and the unit of the functional group equivalent is g/mol.

The viscosity is a viscosity at 25° C., and the unit of the viscosity is mPa·sec. The viscosity is a value measured using the HAAKE MARS 40 rheometer (product name, manufactured by Thermo Fisher Scientific Inc.) under the following conditions: temperature: 25° C.; shear rate: 0.01/sec; sensor: C35 2°/Ti; measurement mode: oscillation mode; and frequency: 0.03 Hz. In addition, in a case where the material is a solid at 25° C., the material is described as “solid” in the table.

The polymeric MDI represents polymethylene polyphenyl polyisocyanate and means a mixture of 4,4′-MDI and high-molecular-weight polyisocyanate.

As shown in Table 1, in the comparative backing member sheet Nos. c01 to c03, c05, and c08 to c10, the loss tangent of the resin in a range of 0° C. to 50° C. is less than 0.06, which does not satisfy the requirements of the present invention. The comparative backing member sheet Nos. c01 to c03, c05, and c08 to c10 had a low attenuation rate and exhibited poor ultrasound attenuation properties. In addition, in the comparative backing member sheet No. c06, the loss tangent of the resin in a range of −20° C. to 110° C. is 1.50 or more, which does not satisfy the requirements of the present invention. The comparative backing member sheet No. c06 exhibited poor workability. In addition, in the comparative backing member sheet No. c04, the loss tangent of the resin in a range of 0° C. to 50° C. is less than 0.06, and the loss tangent of the resin in a range of −20° C. to 110° C. is 1.50 or more, which does not satisfy the requirements of the present invention. The comparative backing member sheet No. c04 exhibited poor ultrasound attenuation properties and workability. In the comparative backing member sheet No. c07, the storage elastic modulus of the backing material in a range of 0° C. to 50° C. is less than 1000 MPa, which does not satisfy the requirements of the present invention. The comparative backing member sheet No. c07 exhibited poor workability. In addition, the comparative backing member sheet No. c17, the content of the resin in the backing material is 20% by volume, which does not satisfy the requirements of the present invention. The comparative backing member sheet No. c17 exhibited poor workability. In the comparative backing member sheet No. c18, the content of the resin in the backing material is 55% by volume, which does not satisfy the requirements of the present invention. The comparative backing member sheet No. c18 exhibited poor workability.

Further, the storage elastic modulus of the backing material in a range of 0° C. to 50° C. is less than 1000 MPa in the comparative backing member sheet Nos. c11 to c16 using a silicone resin, a silicone resin-modified epoxy resin, ethylene-propylene (EPDM) rubber, an ethylene-vinyl acetate copolymer resin, liquid nitrile rubber, or isoprene rubber which is known as a base material constituting the backing material, and the loss tangent of the resin in a range of 0° C. to 50° C. is less than 0.06 in the comparative backing member sheet Nos. cl1, c12, c15, and c16, which do not satisfy the requirements of the present invention. The comparative backing member sheet Nos. c11 to c16 exhibited poor workability.

In contrast, it was found that the backing member sheet Nos. 101 to 125 satisfying the requirements of the present invention exhibited excellent ultrasound attenuation properties and workability.

In addition, in the backing member sheet Nos. 101 to 125 satisfying the requirements of the present invention, the backing member sheet containing no hollow particles also exhibited excellent ultrasound attenuation properties and workability, and the effects of the present invention were obtained.

Although the present invention has been described with reference to the embodiments, it is the intention of the inventors of the present invention that the present invention should not be limited by any of the details of the description unless otherwise specified and rather be construed broadly within the spirit and scope of the invention appended in the claims.

### EXPLANATION OF REFERENCES

1: acoustic lens
2: acoustic matching layer
3: piezoelectric element layer
4: backing member
7: housing
9: cord
10: ultrasound probe

What is claimed is:

1. A backing material for an ultrasound probe, comprising:
thermally conductive particles; and
a resin,
wherein the resin includes any one of the following (A) to (C):
(A) a reaction cured product of an epoxy resin having a polyurethane structure and a polyamine compound;
(B) a reaction cured product of an epoxy resin and a polyamine compound, the reaction cured product having a polyether structure; and
(C) a reaction cured product of a polyisocyanate compound and a polyamine compound,
a loss tangent of the resin in a range of 0° C. to 50° C. is 0.06 or more and a loss tangent of the resin in a range of −20° C. to 110° C. is less than 1.50,
a storage elastic modulus of the backing material in a range of 0° C. to 50° C. is 1000 MPa or more, and
a content of the resin in the backing material is 25% to 50% by volume.

2. The backing material according to claim 1,
wherein the thermally conductive particles include at least one of metal particles or ceramic particles.

3. An ultrasound probe comprising:
the backing material according to claim 1.

4. An ultrasound diagnostic apparatus using the ultrasound probe according to claim 3.

5. A curable resin composition for forming the backing material according to claim 1, the curable resin composition comprising:
the thermally conductive particles; and
any one of the following (a) to (c) as a resin component:
(a) a combination of an epoxy resin having a polyurethane structure and a polyamine compound;
(b) a combination of an epoxy resin and a polyamine compound, at least one of the epoxy resin or the polyamine compound having a polyether structure; and
(c) a combination of a polyisocyanate compound and a polyamine compound.
wherein a viscosity of the curable resin composition is 5000 Pa·sec or less under conditions of 25° C. and a shear rate of 0.01/sec.
